SEQUENCE LISTING (4 sequences)

SEQ ID NO: 1 - DNA, 17 bp, Artificial Sequence
  Description of Artificial Sequence Primer
cgaggtcgacggtatcg

SEQ ID NO: 2 - DNA, 65 bp, Artificial Sequence
  Description of Artificial Sequence Primer
tctagaactagtggatccttttcttttcttgaattctttcttttcttttatcgataccgtcgacc

SEQ ID NO: 3 - DNA, 68 bp, Artificial Sequence
  Description of Artificial Sequence Primer
cgaggtcgacggtatcgataaaagaaaagaaagaattcaagaaaagaaaaggatccactagttctaga

SEQ ID NO: 4 - DNA, 68 bp, Artificial Sequence
  Description of Artificial Sequence Primer
tctagaactagtggatccttttcttttcttgaattctttcttttcttttatcgataccgtcgacctcg

(19) United States
(12) Patent Application Publication
(10) Pub. No.: US 2004/0091602 A1
(43) Pub. Date: May 13, 2004

(54) Method for immobilizing biologically active molecules

(76) Inventors: Hyun Jin Hwang, Seoul (KR); Jeong Hee Kim, Seoul (KR)

Correspondence Address: EDWARDS & ANGELL, LLP, P.O. BOX 55874, BOSTON, MA, 02205, US

(21) Appl. No.: 10/406,155

(22) Filed: Apr. 2, 2003

Related U.S. Application Data
(63) Continuation-in-part of application No. PCT/KR01/01239, filed on Jul. 20, 2001.
(63) Continuation-in-part of application No. PCT/KR00/01104, filed on Oct. 4, 2000.
(60) Provisional application No. 60/369,429, filed on Apr. 2, 2002.

(51) Int. Cl. B05D 3/00; C12Q 1/68; G01N 33/53

(52) U.S. Cl. 427/2.11; 435/6; 435/7.1

(57) ABSTRACT

The present invention relates to a method for immobilizing a biologically active molecule on a supporting material. In one embodiment, the invention relates to an efficient immobilization method that maximally preserves the biological activity of the immobilized molecule by masking the active site of the molecule and optimizing interaction of the masked molecule to the supporting material.

### CROSS-REFERENCE TO RELATED APPLICATIONS

The present application is a continuation-in-part (CIP) of PCT/KR01/01239 as filed on Jul. 20, 2001. The CIP application claims priority to PCT application PCT/KR00/01104 as filed on Oct. 4, 2000 as well as U.S. Provisional Application Serial No.: 60/369,429 as filed on Apr. 2, 2002. The disclosures of the PCT/KR00/01104, PCT/KR01/01239 and 60/369,429 applications are incorporated herein by reference.

### TECHNICAL FIELD

The present invention relates to a method for immobilizing a biologically active molecule on a supporting material. More particularly, the invention relates to an efficient immobilization method that maximally preserves the biological activity of the immobilized molecule by masking the active site of the molecule and optimizing interaction of the masked molecule to the supporting material.

### BACKGROUND

Recently, the effort for identifying and/or probing activities of biologically active molecules such as nucleic acids, proteins, enzymes, antibodies, antigens, and the like by combining various biotechnologies and semiconductor manufacturing technologies is proliferating worldwide. Immobilization of desired biologically active molecules on a small silicon or glass chip within specific areas of micro size and biochemical assay thereafter allow to obtain useful information efficiently. Efficient immobilization methods are required in developing biochips, Lab-on-a-chip, etc. for diagnosis, drug screening, and research, and also in enhancing efficiencies of various biochemical assay processes that include separation, purification, and recycling of biologically active molecules.

Commonly used current methods utilize nonspecific chemical bonding for immobilization of the biologically active molecule. In such methods, a linker molecule having a reaction group is introduced on a substrate material and chemical bonds are formed between multiple reaction groups of the linker molecules and multiple reaction groups of the biologically active molecule. In the immobilization reaction, the biologically active molecule is immobilized on the supporting material through a variety of bonding and binding such as covalent bonding, ionic bonding, coordination bonding, hydrogen bonding, packing, etc. using various reaction groups such as amine, carboxyl, alcohol, aldehyde, thiol, etc., that exist on the surface of the biologically active molecule. Also, the biologically active molecule can have a single or multiple active sites for forming complexes with particular compounds such as substrate, coenzyme, antigen, antibody, etc.

For example, in one of the most utilized immobilization methods, a linker molecule having a reaction group is introduced onto a substrate material by physical or chemical adsorption, and the reaction group of the linker molecule is activated to induce an immobilization reaction with the biologically active molecule. For example, carboxyl can be activated to react with primary amine using 1-ethyl-3-(3-dimethylaminopropyl)carbodiimide (EDC), N-hydroxysuccinimide (NHS) in the presence of EDC, or SOCl₂. Therefore, the biologically active molecule can be immobilized by reacting the activated carboxyl groups of the linker molecules with primary amines on the surface of the biologically active molecule (or protein). (Anal. Biochem., vol. 185, pp. 131-135, 1990; Anal. Chem., vol. 66, pp. 1369-1377, 1994; Biosens. Bioelectron., vol. 11, pp. 757-768, 1996; Biosens. Bioelectron., vol. 12, pp. 977-989, 1997; Science, vol. 289, pp. 1760-1763, 2000).

However, there is increasing recognition that when a biologically active molecule is immobilized on a supporting material by nonspecific chemical bonding as in the above examples, there can be substantial problems.

For instance, since a plurality of reaction groups exist on the surface of the biologically active molecule as well as on the supporting material, a plurality of immobilization bonds can be formed between the biologically active molecule and the supporting material. Such nonspecific formation of multiple immobilization bonds in various regions of the biologically active molecule can induce structural change and destruction of the biologically active molecule upon immobilization, thereby causing substantial reduction or destruction of the activity of the biologically active molecule.

Additionally, nonspecific immobilization bonds can be formed directly at or near the active site of certain molecules. Such chemical bonding at or near the active site can directly damage it, thereby reducing or destroying the activity of the biologically active molecule after immobilization.

Therefore, the immobilization methods using such nonspecific chemical bonding give rise to damage in the active site and the molecular structure change in the biologically active molecule, thereby reducing the activity per immobilized molecule and thus resulting in decrease of the overall activity per unit area of immobilization.

There have been some attempts to develop better immobilization methods. For example, U.S. Pat. No. 4,180,383 discloses a method of making an immobilized immunoadsorbent in which an antigen is used as a masking agent to protect antibody active sites during the conjugation reaction with a polymer support. U.S. Pat. No. 6,194,552 and Subramanian A. and W. Velander (1996) J. of Mol. Recognition 9: 528 also disclose a similar method for preparing an immobilized immunoadsorbent using an antigen as a masking agent. U.S. Pat. No. 6,172,202 (see also PCT/EP93/03429 (WO 94/13322)) discloses a method for preparing a conjugate of a protein (or a glycoprotein) with a water soluble protein using an antibody or an antiidiotypic antibody as a masking agent. However, there is emerging recognition that such methods have drawbacks.

For instance, the methods often require exhaustive and prolonged binding of the masking agent to protect active sites sufficiently before reaction with polymer. Such methods thus may not be suitable when supply of the masking agent is limited or when the antibody or the protein to be protected is sensitive to extended incubation with the masking agent. Moreover, since the masking agent used in above methods is a protein or a polypeptide having the same reaction groups as the antibody or the protein to be immobilized (or conjugated), the masking agent can also be undesirably conjugated to the polymer support.

Related attempts to protect proteins with different types of masking agents during the conjugation reaction have also been reported. For instance, U.S. Pat. Pub. No. 2002/0120109 discloses a method of protecting a protein during the conjugation reaction using negatively charged polymers.

More generally, there is increasing understanding that prior methods of protecting biologically active molecules before reaction with a supporting material have not always been satisfactory. That is, there is doubt that current approaches focusing on direct protection of active sites will be enough to preserve the biological activity of many immobilized molecules. In practice, it has been difficult or even impossible to preserve the biological activity of many molecules following immobilization. It would be desirable to have a method of immobilizing (or conjugating) a biologically active molecule to a supporting (substrate) material that is broadly applicable to a wide spectrum of molecules. It would be further desirable to have methods that protect not only the active sites of such molecules but also optimize interaction with the supporting material to preserve the biological activities of the molecules of interest after immobilization.

### SUMMARY OF THE INVENTION

The present invention features a method for immobilizing a biologically active molecule on a supporting material. The method is broadly applicable to a wide range of different molecules and it can be used to preserve the biological activity of the molecule after immobilization. Preferred invention methods protect (mask) the active site(s) of the biologically active molecule and optimize interaction of the masked molecule to the supporting material.

It has been found that it is possible to preserve the biological activity of a wide spectrum of different molecules by masking the active site(s) and optimizing interaction of the masked molecule to a desired supporting material. More specifically, it has been discovered that by controlling the rate of immobilization of the masked molecule, it is possible to maintain most of the natural biological activity of the molecule following immobilization to the supporting material. In preferred embodiments, the rate of the immobilization reaction is optimized so as to minimize the number of immobilization bonds formed between each masked biologically active molecule and the supporting material. At the same time, the probability of immobilizing the molecule is maximized to the greatest extent possible.

As will be more apparent from the discussion and Examples that follow, Applicants have learned that it is possible to optimize the rate of the immobilization reaction by balancing two competing kinetic parameters in the immobilization reaction. This inventive concept can be appreciated by considering a hypothetical (ideal) immobilization reaction where it would be possible to achieve a theoretical maximum value of the preserved activity per unit area of immobilization on the supporting material if the following two conditions could be fulfilled simultaneously: (1) forming a minimum number of immobilization bonding per biologically active molecule so as not to reduce or eliminate activity of the immobilized molecule and (2) forming a maximum number density of the biologically active molecule at which no or negligible activity reducing effect occurs. However in “real life” immobilization reactions, satisfying these two conditions is difficult or sometimes impossible because they oppose each other. That is, while the rate of the immobilization reaction must be reduced to reduce the number of the immobilization bonding per immobilized molecule, the rate must also be enhanced to increase the number of immobilized molecules.

It is an object of the present invention to provide a solution to this thermodynamic dilemma by identifying a “compromise” or “opportunity window” between the two opposing kinetic parameters. That is, the invention provides specific reaction parameters that optimize both opposing kinetic parameters and allow one to immobilize nearly any biologically active molecule with a highly preserved activity. In preferred embodiments, the immobilized molecule has a kinetically allowed maximum activity per unit area of immobilization on the supporting material.

More specifically, it is a goal of the present invention to provide optimized immobilization reaction conditions that can be characterized as providing: 1) maximum preservation of activity of individual molecules, 2) minimal probability of forming multiple immobilization bonds per immobilized molecule and 3) maximal increase in overall number density of the immobilized molecule (i.e., by maximizing the probability of forming at least one bond per each molecule, within the practical limit). The Examples below show a preferred procedure in which the two opposing kinetic parameters discussed above can be nearly independently controlled or optimized. If desired, the procedure can be readily adapted in accord with this invention to immobilize nearly any biomolecule or fragment thereof in instances where significant preservation of bioactivity in bound form is needed.

It will be apparent that the number of the immobilization bonds per immobilized molecule will often depend more on the number density (and also reactivity) of the reaction group on the supporting material than many other kinetic parameters mentioned herein. By the phrase “number density” is meant the number of entities of interest present per unit area of a surface. For example, the number density of the reaction group on the supporting material means the number of the reaction groups per unit area of the surface of the supporting material. Without wishing to be bound to theory, it is believed that when a molecule is bound to the supporting material by formation of first immobilization bond, formation of additional bonds between the plurality of the reaction groups on the bound molecule and the plurality of the reaction groups on the supporting material becomes “intramolecular”. That binding is generally understood to be much faster than “intermolecular” macroscopic kinetics between the not-yet-bound molecules and the supporting material. The invention provides a solution to this problem, for instance, by providing control over other reaction parameters such as concentration of the molecule to be immobilized, the reaction time, temperature, pH, and optionally a reaction inducing agent such as a coupling agent to increase the overall number density of the immobilized molecule. Preferably, the number of the immobilization bonds per immobilized molecule is minimized by controlling (typically reducing) the number density of the reaction group on the supporting material. Surprisingly, it has been discovered that by controlling and thus optimizing the rate of the immobilization reaction, more than about 20% of the natural biological activity of the subject molecule can be retained, typically more than about 30% or 40% of such activity as compared to the theoretical maximum activity per unit area of immobilization on the supporting material (i.e., compared to the full monolayer amount of immobilized, fully active molecules).

Also without wishing to be bound to theory, it is believed that prior methods of masking biologically active molecules have relied too much on protecting active sites such as those that bind antigen or ligand. This approach, while affording some protection against harmful immobilization reactions, is believed to have left significant portions of the molecule underprotected or exposed. For instance, regions outside the active site (accessory sites) can still be exposed to harmful reaction with the supporting material. There is increasing acknowledgement that multiple bond formation in the accessory sites of many molecules including receptors, enzymes and even certain immune system molecules can profoundly impact active site function. Prior to the present invention, there has been no reliable and broadly applicable way of protecting both the active and accessory sites of these molecules from undesired reaction with the supporting material.

The present invention addresses this need by providing, for the first time, a reliable and generally applicable method of preserving the biological activity of a wide range of biologically active molecules. Such molecules include but are not limited to, antibodies, receptors, enzymes; and biologically active fragments thereof. Preferred practice of the invention involves masking the active site(s) of the molecule and controllably optimizing the rate of immobilization of the masked molecule to the supporting material. Preferably, the rate of immobilization is adjusted such that a minimum number of immobilization bonds are formed per immobilized molecule and at the same time a maximum amount of the masked molecule is bound to the supporting material within the practical limit. Importantly, it has been found that the masking and controlled immobilization steps act synergistically to preserve the biological activity of a wide range of important molecules. That is, the observed activity of a biologically active molecule immobilized according to the invention is surprisingly higher and more robust when compared to results obtained by only masking the molecule or controlling the rate of immobilization of an unmasked molecule. Immobilization results achieved with the invention are also significantly higher when compared to more traditional random immobilization approaches e.g., by at least about 5-fold or more.

Practice of the invention provides important advantages. For example, it has been difficult to immobilize many “sensitive” molecules such as enzymes and receptors using many of the prior art methods. It is believed that use of such methods has unnecessarily exposed sensitive sites outside the active site to damaging immobilization reactions. This has reduced and often eliminated the biologically activity of many sensitive molecules after immobilization. In contrast, preferred practice of the invention is designed specifically to protect active and accessory sites alike, thereby helping to preserve biological activity of the immobilized molecule. Importantly, the invention can be used to preserve the biological activity of a wide range of molecules including enzymes, proteins, factors, receptors, and other sensitive or potentially sensitive biomolecules that heretobefore have been difficult or impossible to immobilize on a supporting material with acceptable efficiency.

Accordingly, and in one aspect, the present invention provides a method for immobilizing a biologically active molecule that preferably does not give rise to steric hindrance or structural change in the active site by means of masking the active site of the biologically active molecule during the immobilization reaction and controlling the rate of immobilization to a supporting material of interest. The present invention therefore provides a method that can improve the activity preservation ratio of the immobilized biologically active molecule, thereby enhancing the overall activity per unit area of immobilization.

The present invention also provides a method for immobilizing a biologically active molecule that is useful in developing biochips or DNA chips.

Furthermore, the present invention provides an immobilized biologically active molecule that represents high activity preservation ratio.

The efficient immobilization method of the present invention can maximally preserve the activity of a biologically active molecule. In one embodiment, the method comprises the steps of: (a) reacting the biologically active molecule with a masking compound that selectively binds to the active site so as to mask the active site; (b) forming a supporting material by controllably introducing on a substrate material a linking group (e.g., a linker) that will bind to the masked biologically active molecule prepared in step (a); (c) controlling the rate of the immobilization reaction in which the masked biologically active molecule prepared in step (a) binds to the linker on the supporting material formed in step (b); and (d) immobilizing the masked biologically active molecule prepared in step (a) on the supporting material by reacting with the linker on the supporting material formed in step (b).

Step (a) of the present invention where the active site of the biologically active molecule is masked, is a step where the masking compound that binds selectively to the active site of the biologically active molecule reacts with the biologically active molecule or with its active site, thereby forming a complex, a masked biologically active molecule. This masking step can be performed before or simultaneously with step (d), where the biologically active molecule is immobilized by reacting with the reaction group of the linker.

Examples of the biologically active molecules include protein, enzyme, receptor, antigen, antibody, and biologically active fragments thereof. The masking compound that can be used for masking the active site of the biologically active molecule, can be selected from the group consisting of substrate, inhibitor, cofactor, their chemically modified compound, their homolog and their derivative for masking enzyme; antibody and its modification for masking antigen; antigen and its modification for masking antibody; and ligand and its modification for masking receptor. For example, an enzyme whose substrate is DNA or RNA can be masked by DNA, RNA, their derivative, or their homolog. Antibody can be masked by antigen or its derivative or homolog, and similarly antigen by antibody or its derivative or homolog; e.g. anti-DNA antibody can be masked by DNA as used in one of the Examples described in the present invention.

The masking compound binds to one or more active sites or cofactor sites of the biologically active molecule to form a complex. The complex can be formed through covalent bonding, ionic bonding, coordination bonding, hydrogen bonding, dipole-dipole interaction, packing, or the combination of two or more of such bonding or binding. The reaction time of complex formation can vary from several seconds to a day. The reaction pH is not specifically limited, as far as the activity of the biologically active molecule is not destructed and complex formation for masking the active site can thus take place efficiently at the given pH. The masking (or protection) ratio, i.e., ratio of the masked amount to total amount of the biologically active molecule, can be selected preferably within the range of between about 5% to about 100%.

Formation of immobilization bonding at or near the active site can be prevented by masking the active site of the biologically active molecule with a masking compound (for example substrate or inhibitor for enzyme) that selectively binds to the active site, as described in step (a).

The biologically active molecule whose active site is masked can be immobilized on the supporting material, that is, a substrate material where a plurality of the reaction groups for immobilization are controllably introduced. The substrate material herein means a material on which a plurality of the reaction groups can be controllably introduced within the size range comparable to the size of the biologically active molecule.

The reaction groups are typically introduced on the surface of the substrate material by forming a thin film of the linker comprising a reaction group. The linker that forms a thin film on the substrate material has a reaction group to bind to the substrate material by covalent bonding, ionic bonding, coordination bonding, hydrogen bonding, packing, or the combination of two or more of such bonding or binding. Examples of the reaction group of the linker that reacts with the substrate material include thiol, sulfide, disulfide, silane such as alkoxysilane and halogen silane, carboxyl, amine, alcohol, epoxy, aldehyde, alkylhalide, alkene, alkyne, aryl, or the combination of two or more of such reaction groups.

The substrate material that can be used for the present invention includes metal such as Au, Ag, Pt, Cu, etc., non-metal such as silicon wafer, glass, silica, and fused silica, semiconductor, oxide of such elements, organic or inorganic macromolecule, dendrimer, polymer of solid or liquid phase, and their mixture. The substrate material can be fabricated to various shape and morphology such as planar, spherical, linear, or porous type, a microfabricated gel pad, a nano-particle, etc. The substrate material can include any material with various shape and morphology, as far as its size is larger than or equal to several nm and it is thus possible to introduce a plurality of the reaction groups for immobilization on its surface. Because the size of the biologically active molecule is on the nm range that is an order of magnitude larger than the atomic distance on the Å range, any substrate material of nm size or larger can be used in the present invention as far as a plurality of the reaction groups can be introduced on its surface.

Examples of the reaction groups of the linker that react with the biologically active molecule include carboxyl, amine, alcohol, epoxy, aldehyde, thiol, sulfide, disulfide, alkyl halide, alkene, alkyne, aryl, or the combination of two or more of such groups. These reaction groups can react to connect the masked biologically active molecule to the supporting material.

The immobilization bonding between the linker and the biologically active molecule can also be covalent bonding, ionic bonding, coordination bonding, hydrogen bonding, or their combination. The immobilization bonding can be amide bonding, imine bonding, sulfide bonding, disulfide bonding, ester bonding, ether bonding, amine bonding, or the combination of two or more of such bonding. For example, amine of the biologically active molecule and carboxyl of the linker or vice versa can react to form amide bonding, amine of the biologically active molecule and aldehyde of the linker or vice versa to form imine bonding, and thiol of the biologically active molecule and thiol of the supporting material to form disulfide bonding.

Even though the active site of the biologically active molecule is masked with the masking compound, structural change in the biologically active molecule could occur to damage or destruct the activity of the immobilized biologically active molecule if too many immobilization bondings can be formed. In this case, reduction in the activity due to formation of the multiple immobilization bondings can be prevented by down-kinetic-regulation, that is, by reducing the rate of the immobilization reaction and thus reducing the probability of the immobilization reaction. However, when down-kinetic-regulation is excessive, the overall activity per unit area of immobilization can decrease due to reduction in the probability of immobilizing the biologically active molecule. Therefore, it is essential to optimize the rate of the immobilization reaction by controlling the kinetic variables such that the probability of forming multiple immobilization bonding for each biologically active molecule is reduced, while keeping the probability of immobilizing the biologically active molecule as high as possible. The reaction rate is optimized in the present invention by controlling number density (or mole fraction) of the reaction group on the substrate material, concentration of the biologically active molecule, pH of the reaction solution, reaction time, reaction temperature, and type of the coupling reagent.

For example, and in one embodiment, the number density (or mole fraction) of the reaction group is controlled in the Examples of the present invention by introducing two different thiol molecules having two different terminal groups onto the surface of a substrate material, Au. One of the thiol molecules has the reaction group for immobilization in its terminal and a longer alkyl chain, while the other has a non-reactive group, different from the reaction group for immobilization, and a shorter alkyl chain. The latter thiol molecule is used to mask or protect the substrate material against the immobilization reaction. The former thiol molecule having the reaction group is selected from the group consisting of mercaptocarboxylic acid such as 12-mercaptododecanoic acid, mercaptoaminoalkane, mercaptoaldehyde, dimercaptoaldehyde, dimercaptoalkane, and sulfide and disulfide having a reaction group such as carboxyl, thiol, alcohol, aldehyde, amine, etc. The latter thiol molecule having the non-reactive group can be selected from the group consisting of mercaptoalcohol such as 6-mercapto-1-hexanol, mercaptoalkane such as 1-heptanethiol, and sulfide or disulfide having a non-reactive group. It is preferable that the thiol molecule having the reaction group is mercaptocarboxylic acid or mercaptoaminoalkane and the thiol molecule having the non-reactive group is mercaptoalcohol or mercaptoalkane; that the former molecule is mercaptoaldehyde and the latter molecule is mercaptoalcohol or mercaptoalkane; and that the former molecule is dimercaptoalkane and the latter molecule is mercaptoalcohol or mercaptoalkane.

In the embodiments using Au as a substrate material, the mole fraction of the linker molecule having the reaction group for immobilization is preferably about 0.05% to about 50%, more preferably about 0.5% to about 30% and most preferably about 0.5% to about 10%. When the mole fraction of the linker molecule having the reaction group is too high, for example in excess of 50%, formation of multiple immobilization bonding can damage the activity of the immobilized biologically active molecule as discussed previously. When it is too low, for example less than 0.5%, the probability of immobilization decreases. Therefore, the overall activity per unit area of immobilization decreases in such too high or too low mole fraction ranges.

The range of the mole fraction of the linker molecule preferable when the substrate material is Au can be converted to range of the number density of the linker or the reaction group that is preferable for other supporting or substrate materials. Maximum number density of the reaction group that can be introduced to the Au surface is known to be about 4×10¹⁴ cm⁻² (4.99 ∈ spacing). Therefore, the number density of the linker or the reaction group on the supporting material is preferably between about 2×10¹¹ cm⁻² to 2×10¹⁴ cm⁻², more preferably about 2×10¹² cm⁻² to about 1.2×10¹⁴ cm⁻², and most preferably about 2×10¹² cm⁻² to about 4×10¹³ cm⁻².

The reaction group introduced on the substrate material, for example carboxyl, can be activated by a coupling reagent, for example 1-ethyl-3-(3-dimethylaminopropyl)carbodiimide (EDC), N-hydroxysuccinimide (NHS) in the presence of EDC, SOCl₂, etc. The activated reaction group then reacts with the masked biologically active molecule.

The concentration of the biologically active molecule for optimizing the immobilization reaction is preferably in the range of between about 0.01 μg/ml to about 10 mg/ml and more preferably between from about 0.1 μg/ml to about 1 mg/ml. The pH of the immobilization reaction is in the range of between about 4 to about 10, and the immobilization reaction time is in the range of several seconds to 24 hours.

The method for immobilizing the biologically active molecule provided in the present invention can further include step (e) where the masking compound that is bound to the active site of the immobilized biologically active molecule is removed. By removing the masking compound and exposing the active site, change in the active site due to binding of the masking compound can be recovered, and thus it is possible to obtain a highly preserved activity for the immobilized biologically active molecule. The masking compound can be removed by heating, hydrolysis, dilution, dialysis, pH change, etc.

In the present invention, the biologically active molecule whose active site are masked, are used and the rate of the immobilization reaction is optimized in order to minimize the number of immobilization bonding per biologically active molecule, while keeping the probability of immobilizing the biologically active molecule as high as possible. This in turn prevents or minimizes damage in the activity of the immobilized biologically active molecule and therefore increases the activity preservation ratio, thereby maximizing the overall activity per unit area of immobilization.

### BRIEF DESCRIPTION OF DRAWINGS

FIG. 1a shows change in the activity of the immobilized Taq DNA polymerase according to the protected (or masked) immobilization method (PIM) of the present invention and the random immobilization method (RIM) of the prior art. The agarose gel fluorescence photographs in this figure show the activity change in each case as a function of the mole fraction of 12-mercaptododecanoic acid in the mixed thiol solution used to introduce the carboxyl group as the reaction group for immobilization.

FIG. 1b is a graph showing the relative activity of the immobilized Taq DNA polymerase according to the PIM of the present invention and the RIM of the prior art, as a function of the mole fraction of 12-mercaptododecanoic acid in the mixed thiol solution used to introduce the carboxyl group on the substrate material.

FIG. 2a is an agarose gel fluorescence photograph of the polymerase chain reaction (PCR) products and it shows the activity of the immobilized Taq DNA polymerase as a function of the active site masking ratio for forming the DNA-Taq DNA polymerase complex.

FIG. 2b is a graph showing the activity change of the immobilized Taq DNA polymerase as a function of the active site masking ratio when a partially double stranded DNA and Taq DNA polymerase form a 1:1 complex.

FIG. 3a is an agarose gel fluorescence photograph of the PCR products showing the activity of the immobilized Taq DNA polymerase as a function of pH of the immobilization reaction.

FIG. 3b is a graph showing the activity change of the immobilized Taq DNA polymerase as a function of pH of the immobilization reaction.

FIG. 4a is an agarose gel fluorescence photograph of the PCR products showing activity of the immobilized Taq DNA polymerase as a function of reaction time.

FIG. 4b is a graph showing the activity change of the immobilized Taq DNA polymerase as a function of immobilization reaction time.

FIG. 5a is an agarose gel fluorescence photograph of the PCR products comparing the activity of the immobilized Taq DNA polymerase and that of the Taq DNA polymerase in solution as a function of number of the PCR cycle.

FIG. 5b is a graph comparing the activity of the immobilized Taq DNA polymerase and that of the solution phase Taq DNA polymerase as a function of number of the PCR cycles.

FIG. 6a is an agarose gel fluorescence photograph of the PCR products and it shows the activity of the immobilized Taq DNA polymerase as a function of total amount of the Taq DNA polymerase used in the immobilization reaction.

FIG. 6b is a graph showing the activity change of the immobilized Taq DNA polymerase as a function of total amount of the Taq DNA polymerase used in the immobilization reaction.

FIG. 7 is a graph showing the activity of the immobilized anti-DNA antibody as a function of mole fraction of 12-mercaptododecanoic acid in the mixed thiol solution used to introduce the carboxyl group as the reaction group for immobilization.

FIG. 8 is a graph showing the activity of the anti-DNA antibody as a function of number of moles of the antigenic double stranded DNA.

### DETAILED DESCRIPTION OF THE INVENTION

As discussed, the present invention features a method for immobilizing a biologically active molecule on one or a combination of desired supporting (substrate) materials. More particularly, the invention relates to an efficient immobilization method that maximally preserves the biological activity of the immobilized molecule by masking the active site of the molecule and preferably reducing interaction of the masked molecule to the supporting material. The invention thus protects the active site and accessory sites from undesired reaction with the supporting material.

As also discussed, practice of the invention is broadly applicable to the wide range of biologically active molecules already disclosed including, but not limited to, polypeptides, proteins, antibodies, receptors, enzymes, cytokines, chemokines, hormones, transcription or translation factors, glycoproteins, nucleic acids, as well as mixtures and biologically active fragments thereof. Reference herein to a “biologically active” fragment of a particular molecule means that the fragment has at least about 70% of the activity of the full-length molecule as determined by a recognized assay for that molecule. Examples of such assays include conventional binding assays for polypeptides, proteins and glycoproteins; radioimmunoassays or Western blots for antibodies, standard receptor binding assays, and enzyme reaction rate analysis, etc.

By the phrase “supporting material” or related term is meant a substrate material (e.g., a suitable polymer or polymer blend) that has reactive groups such as reactive linkers bound thereto. An example of a suitable supporting material is an Au surface to which has been added immobilization reaction groups and particularly a monolayer of thiol molecules formed by using standard Au—S bond formation reactions. See the Examples section and Bain, C. B, infra.

The invention is fully compatible with a variety of suitable substrate (or supporting) materials to which immobilization of the biologically active molecule may be desired. Such substrate materials include those described already as well as a matrix of an affinity column, a synthetic or semi-synthetic carbohydrate, polymer, co-polymer, graft co-polymer, polymer adduct, liposome, lipids, microparticle, microcapsule, emulsion or colloidal gold composition. Additionally suitable substrate materials include soluble synthetic polymers such as poly(ethylene glycol), poly(vinyl pyrrolidone), poly(vinyl alcohol), poly(amino acids), divinylether maleic anhydride, ethylene-maleic anhydride, N-(2-hydroxypropyl)methacrylamide, dextran; and blends thereof. See also the U.S. Pat. No. 6,172,202 and references cited therein for additionally suitable polymers. Also suitable for use with the invention are certain co-polymers, polymer blends, graft co-polymers and polymer adducts that are known to be useful for immobilizing biologically active molecules.

In addition to the variety of suitable masking agents disclosed already, the invention can be used to negatively charged polymer masking agents including those disclosed in published U.S. patent application No. 2002/0120109 and PCT/US01/41298.

Successful practice of the invention can be achieved by performing one or a combination of protection strategies. For instance, the rate of immobilization of the masked molecule to a desired supporting material can be controlled by changing the number density, the reactivity (or the reaction lifetime) of the reaction group on the supporting material, or both. Typically the number density of the reaction group on the supporting material is substantially lower (about 5 to about 100 fold lower) than those of the prior supporting materials. If the reactivity (or the reaction lifetime) of the reaction group is higher (lower), the number density of same should be controlled to a lower (higher) density. The number density and the reactivity of the reaction group are closely related to the number of immobilization bonds formed per immobilized molecule. Therefore, these parameters should be substantially reduced to reduce or avoid formation of multiple immobilization bonds per each immobilized molecule that is harmful for preserving activity of the molecule after immobilization. In embodiments in which a linking group is used to join the biologically active molecule to the substrate material, the number density and the reactivity of the linking group or a portion thereof is controlled and preferably substantially reduced. Other strategies for controlling the rate of immobilization of the masked molecule to the supporting material include adjusting one or more of the concentration or molar amount of the masked molecule to be bound, pH, reaction time, reaction temperature, and type of linking group(s) and coupling reagent(s) used. A particular strategy for controlling the rate of immobilization of a masked molecule can be practiced alone or in combination with at least one other of these specific reaction rate control strategies. Choice of a specific rate controlling strategy will be guided by recognized parameters such as the type of molecule to be immobilized, the amount of immobilized biological activity required, the nature of the masking agent, etc.

As mentioned previously, it is possible to control the rate of immobilization of the masked molecule to a desired substrate material by one or a combination of different strategies in accord with this invention. However, it will often be preferred to control the rate of immobilization by one or a few means such as by controlling the mole fraction (or the number density) and the reactivity of the reaction group on the substrate material. In embodiments in which the immobilization is to be carried out in an automated or semi-automated fashion (such as in the production of a slide, chip or wafer with the immobilized molecule), it will often be useful to control the rate of immobilization by controlling the reactivity of one or more linkers bound to the substrate material.

Thus in one invention embodiment, a desired substrate material is contacted by mixture of linkers that includes at least one reactive linker and at least one non-reactive linker. By the phrase “reactive linker” is meant a polyvalent linking molecule such as has already been disclosed, for instance having two chemically reactive groups, in which one reactive chemical group is bound to the substrate material and another reactive (or reaction) group is generally free to bind to the masked molecule. An “non-reactive linker” (having a non-reactive group) is usually monovalent and can be substantially the same or even different from the reactive linker except that the non-reactive linker will not have a reactive group free to bind to the masked molecule. Thus by contacting the substrate material with the linker mixture, it is possible to adjust the mole fraction of reactive and non-reactive linker and desirably control binding of the masked molecule to the substrate material. This feature of the invention allows the user (or an automated device under control of the user) to select the rate of immobilization of the masked molecule to the substrate material. As discussed, it has been found that by controlling the mole fraction (or the number density) of the reactive linker to between about 0.5% to about 50% (about 2×10¹² cm⁻² to 2×10¹⁴ cm⁻²), preferably about 0.5% to about 30% (about 2×10¹² cm⁻² to about 1.2×10¹⁴ cm⁻²), more preferably about 0.5% to about 10% (about 2×10¹² cm⁻² to about 4×10¹³ cm⁻²), it is possible to maximize biological activity of the immobilized (masked) molecule per unit area of the substrate material. Without wishing to be bound to theory, it is believed that by maintaining the mole fraction (or the number density) of the reactive linker within this preferred range and immobilizing a biologically active molecule whose active site(s) is masked by a masking agent, it is possible to minimize harmful immobilization reaction to the active and accessory sites of many biologically active molecules.

As mentioned, the invention features a method for immobilizing a biologically active molecule. In one embodiment, the molecule has one or more active sites on a supporting (or substrate) material which material has a plurality of reactive linkers each having a reaction group. Preferably, the method includes at least one of and preferably all of the following steps:

(a) combining the biologically active molecule with a masking compound that specifically binds to the active site to form a masked molecule; and

(b) immobilizing the masked molecule prepared in step (a) on the supporting (or substrate) material by reacting the molecule with the reaction groups, the reacting being under controlled conditions whereby the masked molecule binds to an average of less than about two of the reaction groups. Preferably, the number density of the reactive linker on the supporting material is adjusted to between about 2×10¹¹ cm⁻² to about 2×10¹⁴ cm⁻².

Suitable supporting (and substrate) materials, controlled conditions, biologically active molecules and fragments thereof, masking compounds, masking ratios, linkers, linker reaction groups, bonding mechanisms, controlling steps, polymers, co-polymers, polymer blends and other acceptable supporting materials for practicing the forgoing method have already been disclosed herein.

By the phrase “controllably reacting”, “reacting under controlled conditions” or related phrases is meant, went it is intended to refer to immobilization of a desired molecule to the supporting material, performing a reaction under conditions such that the mole fraction (or the number density) of the reactive groups (e.g., as present on a linker) is, for instance, between from about 0.5% to about 50% (about 2×10¹² cm⁻² to 2×10¹⁴ cm⁻²), preferably about 0.5% to about 30% (about 2×10¹² to about 1.2×10¹⁴ cm⁻²), more preferably about 0.5% to about 10% (about 2×10¹² cm⁻² to about 4×10¹³ cm⁻²). Typically preferred controlled reactions bind a masked and biologically active molecule of interest to less than about two or three suitable reactive groups, preferably about one of same.

Generally, a maximum of about 400 reaction groups can be introduced on about a 10 nm diameter area of the substrate such as Au. Therefore, if the size of the biologically active molecule to be immobilized is about 10 nm diameter which is similar to the size of the Taq DNA polymerase and most antibodies, an average of one reaction group will be available for each molecule at about 0.25% mole fraction (or about 1×10¹² cm⁻² number density). For smaller molecules, a higher mole fraction (or number density) will be needed to provide average of one reaction group to each molecule, for instance, about 25% molecule fraction (or about 1×10¹⁴ cm⁻² number density) for about 1 nm diameter molecule and about 1% mole fraction (or about 4×10¹² cm⁻² number density) for about 5 nm diameter molecule. Moreover, in many available reaction conditions (especially in aqueous solution), the reaction probability of the reaction group is substantially lower than 100%. Therefore, about 0.5% mole fraction (or about 2×10¹² cm⁻² number density) would be a reasonably lower limit that gives a wide enough range to practice most invention embodiments.

In some invention embodiments, use of a non-reactive linker may not always be necessary. That is, use of the reactive linker alone can also give desired results if the number density of the reactive linker (or the reaction group) is controllably introduced to the support material within the preferred range described above, i.e., between about 2×10¹² cm⁻² to 2×10¹⁴ cm⁻², preferably about 2×10¹² cm⁻² to about 1.2×10¹⁴ cm⁻², more preferably about 2×10¹² cm⁻² to about 4×10¹³ cm⁻².

In some other embodiments, the substrate material itself may be used as a supporting material if the support material already has the reactive linker or the reaction group with its number density controlled or maintained within the preferred range described above.

By the phrase “specific binding” or a related phrase is meant formation of a complex between two or more molecules, preferably two, that is essentially mutually exclusive as determined by standard binding tests including radioimmunoassay, gel assay, centrifugation sedimentation, Western blot, etc. Thus a masking agent in accord with the invention will “specifically bind” a biologically active molecule of interest if it forms a complex with that molecule and no other as determined by the standard binding tests.

The present invention is explained in detail using the following examples, though the examples are only illustrative but not limiting the scope of the present invention.

All references disclosed herein are incorporated by reference.

### EXAMPLE 1

### Immobilization of Taq DNA Polymerase

a) Masking of the Active Site of Taq DNA Polymerase

Taq DNA polymerase was purchased from Perkin Elmer (AmpliTaq Gold™). This DNA polymerase is an chemically modified enzyme with molecular weight of 94 kDa consisting of 832 amino acids that can be activated by heating, for example by placing for 10 minutes at 95° C.

A 65 base single stranded DNA (ss-DNA) (SEQ ID NO: 1) and the KS primer (SEQ ID NO: 2) shown below was mixed in an aqueous buffer solution at 1:1 molar ratio, and the resulting solution was incubated for 10 minutes at 94° C. and was then cooled down slowly below 35° C. in a period of about 1 to 2 hours. During this process, the 65 base ss-DNA (SEQ ID NO: 1) and the KS primer (SEQ ID NO: 2) were annealed to generate a partially double stranded DNA. A desired amount of the Taq DNA polymerase was then added to this solution and the resulting mixture was incubated in a dry bath at 72° C. for 10 minutes. The mixture was then moved to a dry bath at 50° C. and incubated for 20 minutes to prepare the reaction solution of the masked Taq DNA polymerase. In the masked Taq DNA polymerase, Taq DNA polymerase is bound to the 3′ terminal region of the short KS primer of the partially double stranded DNA, where the DNA structure changes from a double strand to a single strand (See S. H. Eom, J. Wang, T. A. Steitz, Nature, vol.382, pp.278-281, 1996). This leads to masking of the active site of the Taq DNA polymerase. The 65 base ss-DNA (SEQ ID NO: 1) and the KS primer (SEQ ID NO: 2) used in this process were synthesized using a DNA synthesizer. The optimal pH for masking the active site was found to be 8.3, at which the activity of the Taq DNA polymerase was known to be the highest.

| | |
|---|---|
| KS primer | |
| 5′ CGAGGTCGACGGTATCG 3′ | (SEQ ID NO: 2) |
| 3′ CCAGCTGCCATAGCTATTTTCTTTTCTTTCTTAAGTTCTTTTCTTTTCCTAGGTGATCAAGATCT 5′ | (SEQ ID NO: 1) |

b) Formation of the Monolayer of Thiol Molecules on the Surface of the Au Substrate and Introduction of the Reaction Group

The Au substrate used was a glass plate of 3.0 mm×5.0 mm size on which Au was vacuum-deposited to about 1000 Å thickness. In order to ensure the cleanness of the surface of the Au thin film, it was washed with Piranha solution for about 10 to 15 minutes at about 60 to 70° C. right before using, and it was rinsed with deionized water and subsequently with absolute ethanol.

In order to introduce the immobilization reaction groups on the Au surface, a monolayer of thiol molecules was formed on the Au surface by using the Au—S bond formation reaction, that is, by using the thiolate formation reaction between the linker having a thiol group and Au, to prepare a supporting material (C. B. Bain, E. B. Troughton, Y.-T. Tao, J. Evall, G M. Whitesides, and R. G Nuzzo, J. Am. Chem. Soc., vol.111, pp.321-335, 1989). In this step, the mixed solution of two kinds of thiol molecules each having an immobilization reaction group and a non-reactive group was used. The mole fraction of the thiol molecule having the immobilization reaction group was controlled by changing its mole fraction in the range of about 0 to 100%, in order to control the mole fraction of the immobilization reaction group on the substrate material. In order to introduce a carboxyl immobilization reaction group, 12-mercaptododecanoic acid with a relatively longer alkyl chain was used. As a thiol molecule having a non-reactive group, 6-mercapto-1-hexanol was used. The Au thin film was placed in 100 μl of a 2 mM mixed thiol solution in ethanol for 2 hours at room temperature to introduce the carboxyl reaction group, and it was then washed with absolute ethanol.

Since the immobilization reaction groups are spatially separated and protrudes out from the surface of the substrate material in the present example, motion of the immobilized biologically active molecule becomes relatively un-restricted and also molecular interactions between the immobilized biologically active molecule and the supporting material can be minimized, leading to increased activity preservation ratio.

c) Activation of the Carboxyl Reaction Group on the Monolayer of the Thiol Molecules

The Au thin film where the carboxyl reaction groups were introduced was placed in 120 μl of an ethanol solution containing 10 mM of 1-ethyl-3-(3-dimethylaminopropyl)carbodiimide (EDC) and 5 mM of N-hydroxysuccinimide (NHS) for 2 hours at room temperature to activate the carboxyl group. The carboxyl group reacts with NHS in the presence of EDC to form NHS-ester (Z. Grabarek and J. Gergely, Anal. Biochem., vol.185, pp.131-135, 1990), thereby being activated.

d) Immobilization Reaction of Taq DNA Polymerase

After activating the carboxyl group on the monolayer, the Au substrate was moved to the solution of the masked Taq DNA polymerase for immobilization reaction. In this step, the activated carboxyl (NHS-ester) on the monolayer reacted with the primary amine (—NH₂) of the protein to form amide bond (—CO—NH—) (Z. Grabarek and J. Gergely, Anal. Biochem., vol. 185, pp.131-135, 1990; V. M. Mirsky, M. Riepl, and O. S. Wolfbeis, Biosens. Bioelectron., vol.12, pp977-989, 1997). As a result, the Taq DNA polymerase was immobilized on the substrate material. The immobilization reaction was carried out at different conditions by varying concentration of the DNA polymerase, pH, reaction time, reaction temperature, etc.

### EXAMPLE 2

### Immobilization of Anti-DNA Antibody

a) Masking of the Active Sites of Anti-DNA Antibody

The anti-DNA antibody is a monoclonal antibody of IgG2b (Chemicon International Inc., cat. No. MAB3032) that recognizes both single and double stranded DNA. It was prepared from mouse ascites by using the calf thmyus DNA as an immunogen. The total protein concentration of this antibody solution as purchased is 25 g/L and about 10% of the protein is anti-DNA antibody.

A 68 bp double stranded DNA (ds-DNA) labeled with ³⁵S, and the anti-DNA antibody were mixed at an appropriate ratio and the resulting solution was incubated for 30 minutes at 37 C to prepare the masked anti-DNA antibody. The sequence of the 68 bp ds-DNA (SEQ ID NO: 3) is given below. The amount of the anti-DNA antibody used was 33 fmol, and that of the 68 bp ds-DNA used for masking the active sites was 2˜120 fmol. The MES buffer at pH 6.0 was used in this masking reaction. The 68 bp ds-DNA labeled with a ³⁵S β emitter was prepared by PCR by adding about 2% mole fraction of α-³⁵S-dATP relative to the total dNTP.

| | |
|---|---|
| KS primer | |
| 5′ CGAGGTCGACGGTATCGATAAAAGAAAAGAAAGAATTCAAGAAAAGAAAAGGATCCACTAGTTCTAGA 3′ | (SEQ ID NO: 3) |
| SK primer | |
| 3′ GCTCCAGCTGCCATAGCTATTTTCTTTTCTTTCTTAAGTTCTTTTCTTTTCCTAGGTGATCAAGATCT 5′ | |

b) Formation of the Monolayer of Thiol Molecules on the Surface of the Au Substrate and Introduction of the Reaction Groups

The Au substrate used was a glass plate of 12.7 mm×12.7 mm size on which Au was vacuum-deposited to about 1000 Å thickness. In order to ensure the cleanness of the surface of the Au thin film, it was washed with Piranha solution for about 10 to 15 minutes at about 60 to 70° C. right before using and was rinsed with deionized water and subsequently with absolute ethanol.

As a thiol molecule having a non-reactive group, 1-heptanethiol was used. A mixed monolayer of 12-mercaptododecanoic acid and 1-heptanethiol was formed as in Example 1. A 9 mm diameter portion of the Au thin film was exposed to 300 μl of a 2 mM mixed thiol solution in ethanol for 2 hours at room temperature to introduce the carboxyl reaction group, and it was then washed with absolute ethanol.

c) Activation of the Carboxyl Reaction Group on the Monolayer of the Thiol Molecules

The Au thin film where the carboxyl reaction groups were introduced was placed in 300 μl of a buffer solution (pH 6.0 MES buffer) containing 10 mM of 1-ethyl-3-(3-dimethylaminopropyl)carbodiimide (EDC) and 5 mM of N-hydroxysulfosuccinimide (sulfo-NHS) for 2 hours at room temperature, with a 9 mm diameter portion of the Au film exposed. The carboxyl group was reacted with sulfo-NHS in the presence of EDC to form sulfo-NHS-ester (J. V. Staros, R. W. Wright, and D. M. Swingle, Anal. Biochem., vol.156, pp.220-222, 1986), thereby being activated.

d) Immobilization Reaction of Anti-DNA Antibody

After activating the carboxyl group on the monolayer, the reaction solution was removed and the Au substrate was placed in the solution of the masked anti-DNA antibody to carry out the immobilization reaction. The total amount of the anti-DNA antibody used was about 33 fmol. In this step, the activated carboxyl (sulfo-NHS-ester) on the supporting material reacted with the primary amine (—NH₂) of the protein to form amide bond (—CO—NH—) (J. V. Staros, R. W. Wright, and D. M. Swingle, Anal. Biochem., vol.156, pp.220-222, 1986; V. M. Mirsky, M. Riepl, and O. S. Wolfbeis, Biosens. Bioelectron., vol.12, pp.977-989, 1997). As a result, the protein was immobilized on the substrate material. The immobilization reaction was carried out in the MES buffer at pH 6.0 for 2 hours at 10° C. The MES buffer contained the ³⁵S labeled 68 bp ds-DNA used to mask the active sites. About 33 fmol of the anti-DNA antibody and about 30 fmol of the 68 bp ds-DNA used for masking the active sites were added to 100 μl of the immobilization reaction solution.

### EXAMPLE 3

### Measurement of the Activity of the Immobilized Taq DNA Polymerase

In order to measure the activity of the immobilized Taq DNA polymerase, PCR was carried out and the amount of the amplified DNA was quantified. PCR was carried out in a Model 480 PCR thermal cycler of Perkin Elmer.

The 65 base ss-DNA (SEQ ID NO: 1) shown in Example 1 was used as a template, and the KS primer (SEQ ID NO: 2) and the SK primer (SEQ ID NO: 4) were used as primers for PCR. The volume of the PCR solution used was 50 μl, and 25 fmol of the 65 base ss-DNA (SEQ ID NO: 1) and 10 pmol each of the KS primer (SEQ ID NO: 2) and the SK primer (SEQ ID NO: 4) were added. As a buffer solution, the pH 8.3, 10× buffer purchased from Perkin Elmer was used after diluting 10 times. The temperature cycle was set as follows:

Hot start step: 94° C., 10 minutes

PCR cycle (20-45 cycles): 94° C., 30 s; 50° C., 60s; 72° C., 30 s

For quantification of the DNA amplified by the PCR, 20 μl of the PCR solution was sampled and analyzed by agarose gel electrophoresis. The PCR products were visualized by fluorescence from ethidium bromide staining, and the PCR product bands were quantified with a densitometer.

### EXAMPLE 4

### Activity of the Immobilized Taq DNA Polymerase as a Function of the Mole Fraction of the Carboxyl Reaction Group

The immobilization reaction was carried out in pH 8.3 phosphate buffer for 30 minutes at 50° C. 0.75 pmol of the Taq DNA polymerase and 1.5 pmol of the masking DNA were added to 50 μl of the immobilization reaction solution. 0.75 pmol of the Taq DNA polymerase corresponds to the amount that can form three monolayers on the area of 3 mm×5 mm of the Au substrate. Thirty-five PCR cycles were carried out with the immobilized Taq DNA polymerase and the resulting activity was measured.

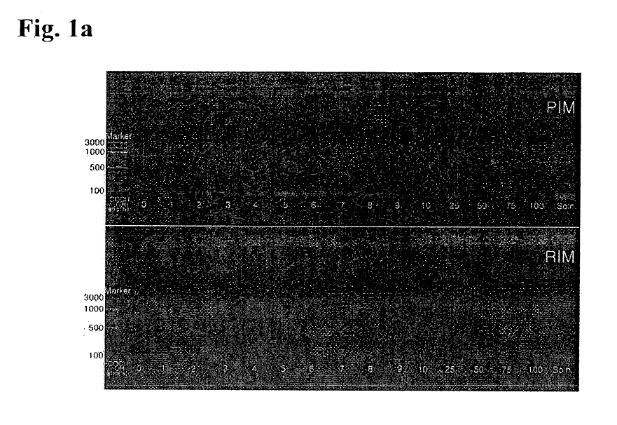

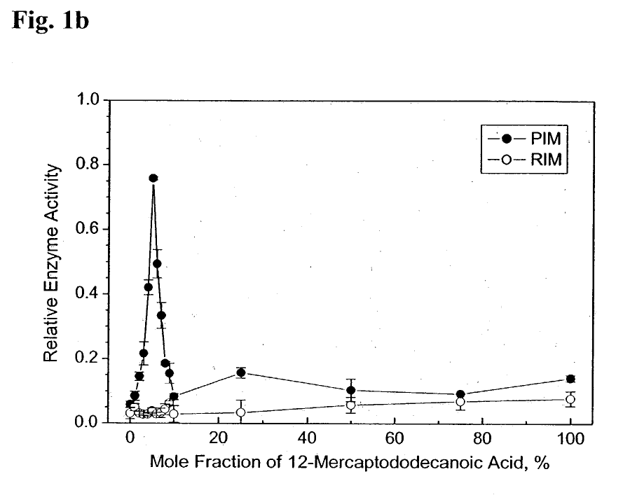

FIG. 1a shows agarose gel fluorescence photographs of the PCR products. The leftmost lanes show ds-DNA molecular weight marker, and the rightmost lanes show the PCR products amplified with one monolayer amount of the Taq DNA polymerase in solution phase. The other lanes show the PCR products resulted from the immobilized Taq DNA polymerase. The number at the bottom of each lane is the mole fraction of 12-mercaptododecanoic acid relative to the total amount of the thiol molecules used.

The activity obtained from the fluorescence photographs of FIG. 1a is shown in FIG. 1b. The x-axis is the mole fraction of the thiol molecule having the carboxyl reaction group, relative to the total moles of the thiol molecules used. The y-axis is the relative activity of the immobilized Taq DNA polymerase, as compared to the activity of one monolayer amount of the solution phase Taq DNA polymerase. The solid circles denote the results of immobilization when the active site was masked (PIM) and the open circles denote those of immobilization when the active site was not masked (RIM).

In the whole range of the mole fraction, the PIM in which the active site was masked shows higher activity than the RIM in which the active site was not masked. Also, it can be seen that the activity of the masked DNA polymerase is the highest when the mole fraction is about 5%. This demonstrates that the activity preservation of the masked DNA polymerase can be maximized kinetically by controlling the mole fraction of the carboxyl reaction group on the substrate material. This result shows that the activity of the immobilized enzyme can be maximized by masking the active site and also by kinetically preventing formation of multiple immobilization bonding that causes reduction or damage of the activity.

### EXAMPLE 5

### Activity of the Immobilized Taq DNA Polymerase as a Function of the Masking Ratio of the Active Site

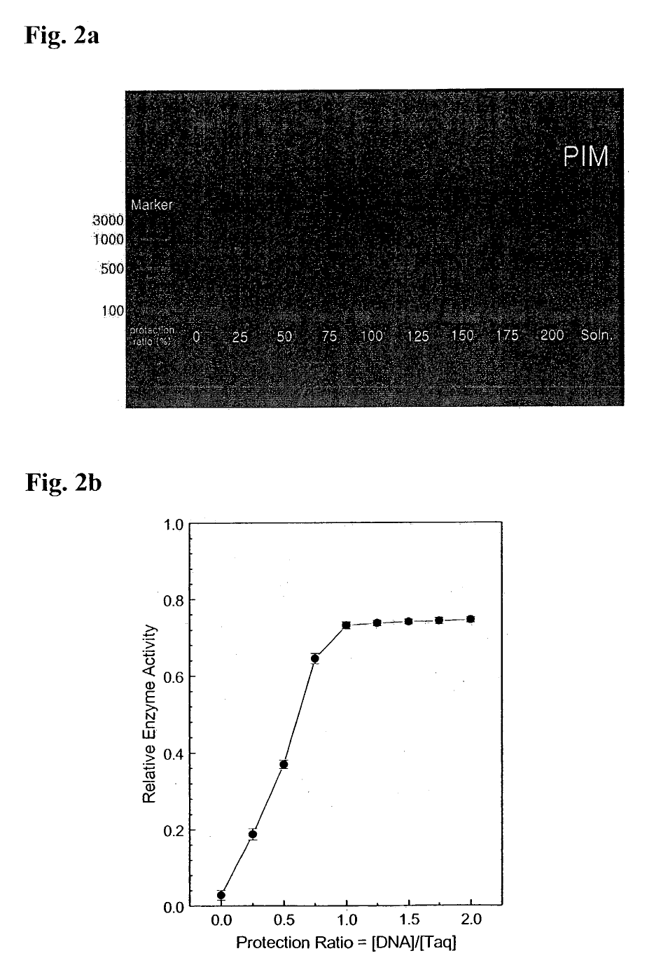

The number of moles of the partially double stranded DNA used to mask the active site relative to that of the Taq DNA polymerase used was varied from 0 to 2, and the activity of the immobilized Taq DNA polymerase was measured. The results are shown in FIGS. 2a and 2b. In FIG. 2a, the leftmost and rightmost lanes are the same as in FIG. 1a, and the other lanes are the results of the PCR products amplified with the immobilized Taq DNA polymerase at different masking ratio. The numbers given below are the % ratio corresponding to the number of moles of the partially double stranded DNA used for masking relative to that of the Taq DNA polymerase.

The activity of the immobilized enzyme is shown as a relative activity with respect to the activity in the solution phase as in FIG. 1b. The molar amount of 12-mercaptododecanoic acid with respect to the total moles of the thiol molecules used for introducing the carboxyl reaction group on the Au surface was 5.0%. The total amount of the Taq DNA polymerase used for the immobilization reaction was 0.75 pmol that corresponded to three monolayers as in FIG. 1b. The other reaction conditions for immobilization and PCR were the same as in Example 4. FIGS. 2a and 2b demonstrate that the active site masking occurs by forming a 1:1 complex of the partially double stranded DNA and the Taq DNA polymerase.

### EXAMPLE 6

### Activity of the Immobilized Taq DNA Polymerase as a Function of the Immobilization pH

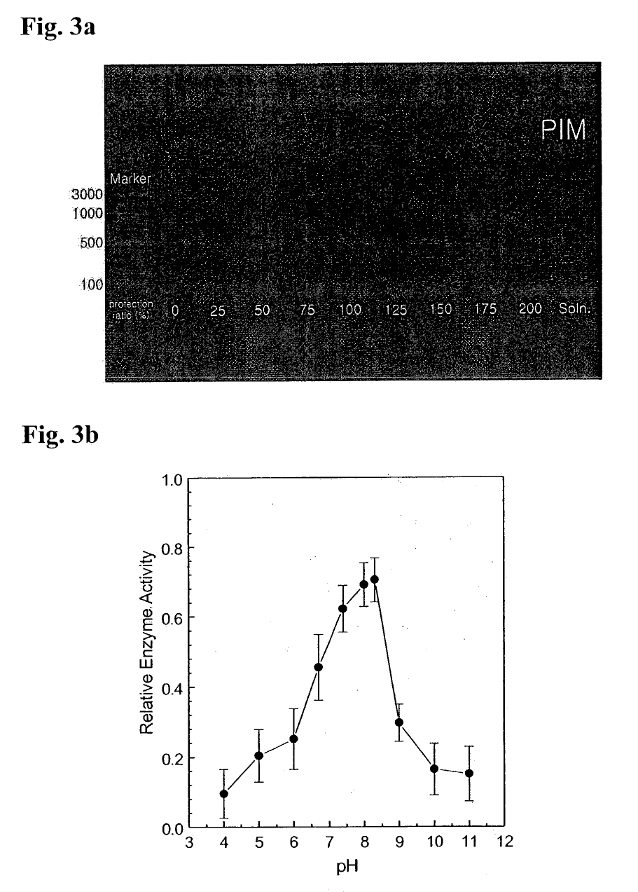

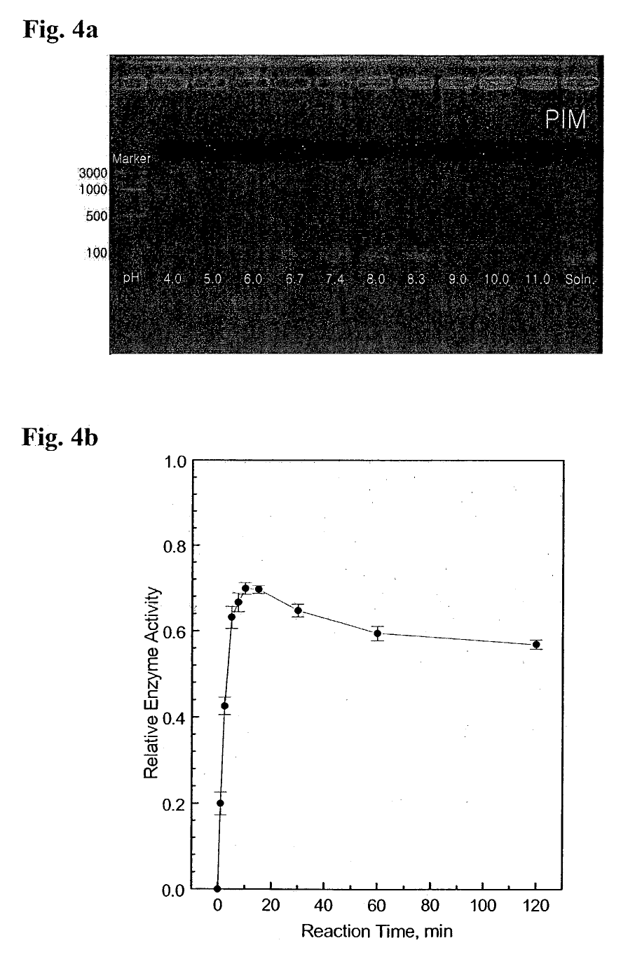

The activity of the immobilized DNA polymerase was measured at different immobilization pH, while keeping the mole fraction of 12-mercaptododecanoic acid at 5.0% with respect to the total moles of the thiol molecules used for introducing the carboxyl reaction group on the Au surface. The other reaction conditions for immobilization and PCR were the same as in Example 4. The results are shown in FIGS. 4a and 3b. The leftmost and rightmost lanes in FIG. 4a are the same as in FIG. 1a, and the other lanes are the results of the PCR products amplified with the immobilized Taq DNA polymerase at different immobilization pH. The pH of the buffer solution used in the immobilization reaction are shown on the bottom of each lane. FIGS. 4a and 3b show that the masking efficiency of the active site is maximized at pH 8.3 where the binding efficiency of the Taq DNA polymerase is known to be maximum.

### EXAMPLE 7

### Activity of the Immobilized Taq DNA Polymerase as a Function of the Immobilization Reaction Time

The activity of the immobilized DNA polymerase was measured at different immobilization reaction time, while keeping the mole fraction of 12-mercaptododecanoic acid at 5.0% with respect to the total moles of the thiol molecules used for introducing the carboxyl reaction group on the Au surface. The other reaction conditions for immobilization and PCR were the same as in Example 4. The results are shown in FIG. 4b.

The rapid increase observed at the reaction time shorter than about 10 minutes in FIG. 4b suggests that the probability of immobilizing Taq DNA polymerase increases with the reaction time while the probability of forming multiple immobilization bonding is kept low. The slow decrease in the region of the reaction time longer than about 10 minutes results from reduction in the activity due to the formation of multiple immobilization bonding as well as the spatial restriction caused by increased number density of the immobilized enzyme. These results suggest that the overall activity per unit area of immobilization can be maximized by optimizing the immobilization reaction time, which is of particular importance kinetically, thereby achieving both high probability of immobilization and suppression of the probability of forming multiple immobilization bonding.

### EXAMPLE 8

### Comparison of Solution Phase and Immobilized Taq DNA Polymerase as a Function of Number of PCR Cycles.

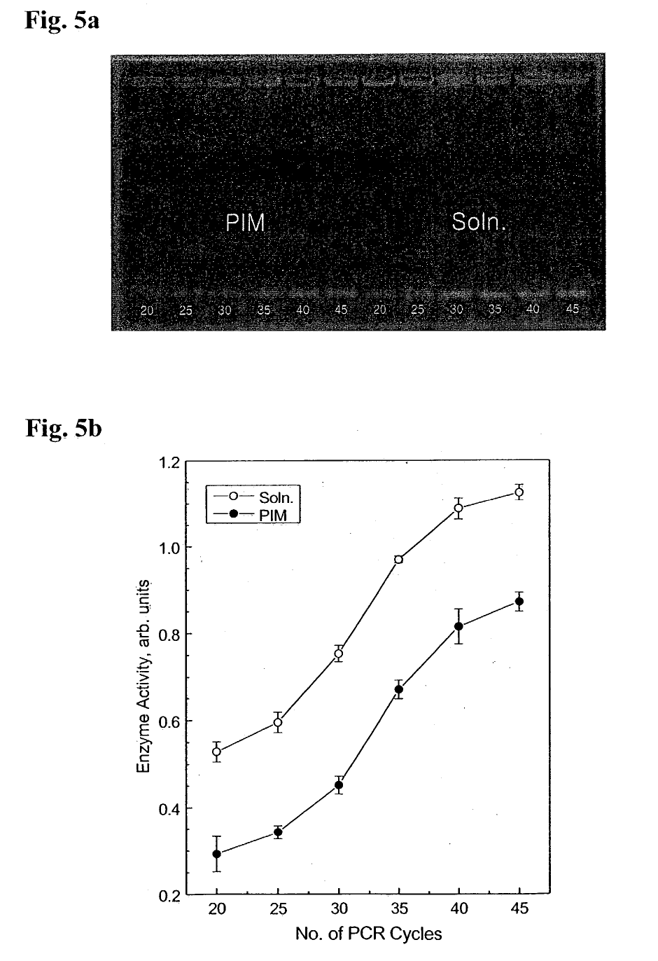

The activity of the immobilized DNA polymerase was measured at different number of PCR cycles, while keeping the mole fraction of 12-mercaptododecanoic acid at 5.0% with respect to the total moles of the thiol molecule used for introducing the carboxyl reaction group on the Au surface. The other reaction conditions for immobilization and PCR were the same as in Example 4. The results are shown in FIGS. 5a and 5b. In FIG. 5a, the number of PCR cycles is given at the bottom of each lane.

FIGS. 5a and 5b show that the trend observed in the activity of the immobilized Taq DNA polymerase is nearly identical to that of the solution phase Taq DNA polymerase. This suggests that the activity preservation ratio per immobilized molecule is maximized, i.e., the activity of the immobilized enzyme being close to the solution phase.

### EXAMPLE 9

### Activity of the Immobilized Taq DNA Polymerase as a Function of Total Amount of Taq DNA Polymerase Used

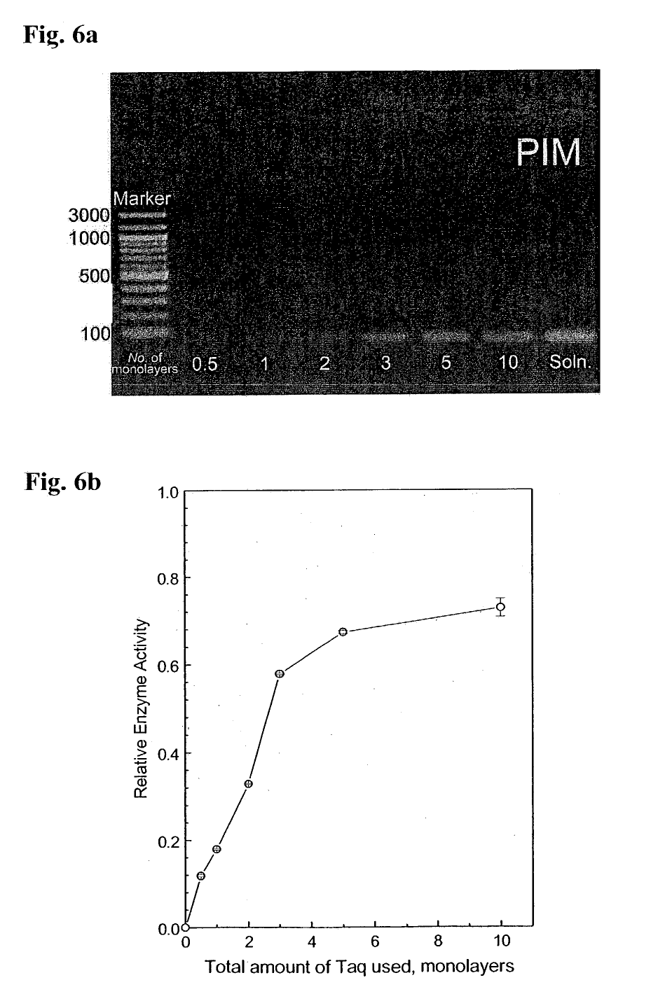

The activity of the immobilized DNA polymerase was measured at different amount of Taq DNA polymerase corresponding to 0 to 10 monolayers, while keeping the mole fraction of 12-mercaptododecanoic acid at 5.0% with respect to the total moles of the thiol molecules used for introducing the carboxyl reaction group on the Au surface. The number of moles of the partially double stranded DNA used for masking the active site was twice that of the Taq DNA polymerase. The other reaction conditions for immobilization and PCR are the same as in Example 4. The results are shown in FIGS. 6a and 6b. The leftmost and rightmost lanes are the same as in FIG. 1a, and the other lanes are the results of the PCR products for different amount of Taq DNA polymerase used. The amount of Taq DNA polymerase is shown in the unit of monolayer at the bottom of each lane.

FIGS. 6a and 6b show that the activity of the immobilized enzyme can be increased by controlling the amount of the Taq DNA polymerase used.

### EXAMPLE 10

### Measurement of Activity of the Immobilized Anti-DNA Antibody

The activity of the immobilized anti-DNA antibody was measured using a β-counter (Beckman, Model LS6500) by counting β-emission from the ³⁵S labeled 68 bp ds-DNA used for masking the active sites. The β-emission measurements were performed with the antibody immobilized Au film placed in 2 ml of the scintillation cocktail.

### EXAMPLE 11

### Activity of the Immobilized Anti-DNA Antibody as a Function of the Mole Fraction of the Carboxyl Reaction Group on the Substrate Material

As in the case of the Taq DNA polymerase, in the whole range of the mole fraction, the PIM in which the active sites were masked shows higher activity than the RIM in which the active sites were not masked. Also it can be seen that the activity of the PIM is the highest when the mole fraction is about 8%. This demonstrates that the activity preservation of the masked antibody can be maximized kinetically by controlling the mole fraction of the carboxyl reaction group on the substrate material. This results show that the activity of immobilized antibody can be maximized by masking the active site and also by kinetically preventing formation of multiple immobilization bonding that causes reduction or damage of the activity.

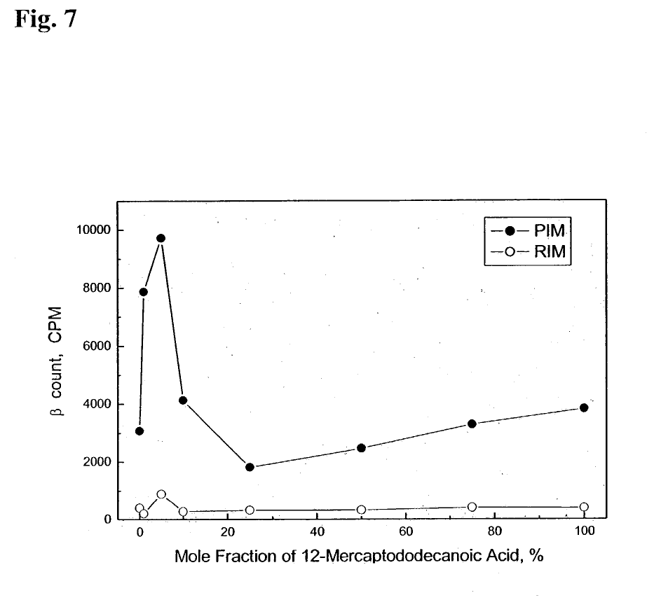

The x-axis in FIG. 7 is the same as that in FIG. 1b, and the y-axis is the activity of the immobilized antibody that is measured by detecting β-emission from the ³⁵S labeled ds-DNA bound to the antibody. The solid circles denote the results of immobilization when the active sites were masked (PIM) and the open circles denote those of immobilization when the active sites were not masked (RIM).

### EXAMPLE 12

### Activity of the Immobilized Anti-DNA Antibody as a Function of the Concentration of the Antigenic ds-DNA.

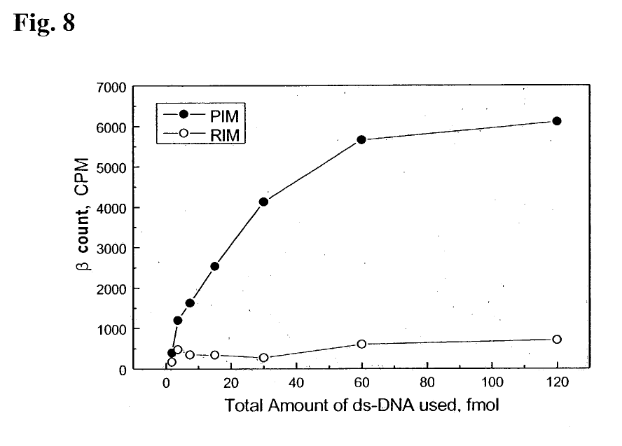

The change in the activity of the immobilized anti-DNA antibody as a function of the concentration of the ³⁵S labeled 68 bp ds-DNA is shown in FIG. 8. The activity of the immobilized anti-DNA antibody was measured at different concentrations of the 68 bp ds-DNA used for masking. The total amount of the anti-DNA antibody used for immobilization reaction was about 33 fmol. The mole fraction of the 12-mercaptododecanoic acid used to introduce carboxyl reaction group on the Au surface with respect to the total moles of the thiol molecules was 10%. The other reaction conditions for immobilization are the same as in Example 11, except for the number of moles of the 68 bp ds-DNA.

In FIG. 8, the solid and open circles denote the PIM and the RIM, respectively. The PIM case shows higher activity than the RIM. The saturation phenomenon was observed in the PIM case when the molar ratio of the anti-DNA antibody to the 68 bp ds-DNA used for masking was in the range 1:1˜1:2. This demonstrates that the active sites were masked by formation of the antigen-antibody complex.

The invention has been described with reference to preferred embodiments thereof. However, it will be appreciated that those skilled in the art, upon consideration of this disclosure, may make modifications and improvements within the spirit and scope of the invention.

All references disclosed herein are incorporated by reference. In particular, co-pending application serial number ______ by Hwang, Hyun Jin and Kim, Jeong Hee entitled “Immobilized DNA Polymerase” as filed on Apr. 2, 2003 is specifically incorporated by reference.

What is claimed is:

1. A method for immobilizing a biologically active molecule on a substrate material comprising the steps of:
(a) reacting the biologically active molecule with a masking compound that selectively binds to the active site so as to mask the active site;
(b) forming a supporting material by controllably introducing on the substrate material a linker that will bind to the masked biologically active molecule prepared in step (a);
(c) controlling the rate of the immobilization reaction in which the masked biologically active molecule prepared in step (a) binds to the linker on the supporting material formed in step (b); and
(d) immobilizing the masked biologically active molecule prepared in step (a) on the supporting material by reacting with the linker on the supporting material formed in step (b).

2. The method of claim 1, wherein step (b) comprises a step of forming a thin film of the linker and a step of controlling the mole fraction (or the number density) of the reaction group of the linker on the supporting material by controlling the ratio of the linker having the reaction group to a non-reactive linker having a non-reactive group.

3. The method of claim 1, wherein step (c) comprises a step of controlling concentration of the masked biologically active molecule.

4. The method of claim 1, wherein step (c) comprises a step of controlling pH.

5. The method of claim 1, wherein step (c) comprises a step of controlling reaction time.

6. The method of claim 1, wherein step (c) comprises a step of controlling reaction temperature.

7. The method of claim 1, which further comprises a step of activating the reaction group of the linker by using a coupling reagent.

8. The method of claim 1, wherein the biologically active molecule is protein, enzyme, antigen, or antibody.

9. The method of claim 1, wherein the making compound that selectively binds to the active site is one selected from the group consisting of substrate, inhibitor, cofactor, or their chemically modified compound, their homolog, and their derivative for masking enzyme; or it is one selected from the group consisting of corresponding antibody, antigen, and their modifications for masking antigen or antibody.

10. The method of claim 1, wherein the active site is one or more active sites or one or more cofactor sites of the biologically active molecule.

11. The method of claim 1, wherein the masking compound that selectively binds to the biologically active molecule binds through covalent bonding, ionic bonding, coordination bonding, hydrogen bonding, dipole-dipole interaction, packing, or their combination.

12. The method of claim 1, wherein the masking ratio of the biologically active molecule is between about 5 to about 100%.

13. The method of claim 1, wherein the substrate material is metal, non-metal, semiconductor, oxide of these elements, organic or inorganic macromolecule, dendrimer, or their mixture; and it is of a planar type, a spherical type, a linear type, a porous type, a microfabricated gel pad, or a nano-particle.

14. The method of claim 1, wherein the linker in step (b) forms a thin film of the linker on the substrate material through covalent bonding, ionic bonding, coordination bonding, hydrogen bonding, packing, or their combination.

15. The method of claim 14, wherein the reaction group of the linker that reacts with the substrate material are thiol, sulfide, disulfide, silane, carboxyl, amine, alcohol, aldehyde, epoxy, alkyl halide, alkene, alkyne, aryl, or their combination.

16. The method of claim 1, wherein the reaction group of the linker that reacts with the biologically active molecule is carboxyl, amine, alcohol, aldehyde, epoxy, thiol, sulfide, disulfide, alkyl halide, alkene, alkyne, aryl, or their combination.

17. The method of claim 1, wherein the biologically active molecule and the reaction group of the linker are connected by covalent bonding, ionic bonding, coordination bonding, hydrogen bonding, packing, or their combination.

18. The method of claim 1, wherein the biologically active molecule and the reaction group of the linker are connected by amide bonding, imine bonding, sulfide bonding, disulfide bonding, ester bonding, ether bonding, amine bonding, or their combination.

19. The method of claim 18, wherein an amine group of the biologically active molecule and a carboxyl group of the linker are connected by amide bonding.

20. The method of claim 18, wherein a carboxyl group of the biologically active molecule and an amine reaction group of the linker are connected by amide bonding.

21. The method of claim 18, wherein an amine group of the biologically active molecule and an aldehyde reaction group of the linker are connected by imine bonding.

22. The method of claim 18, wherein an aldehyde group of the biologically active molecule and an amine reaction group of the linker are connected by imine bonding.

23. The method of claim 18, wherein a thiol group of the biologically active molecule and a thiol reaction group of the linker are connected by disulfide bonding.

24. The method of claim 2, wherein the linker having the reaction group is one selected from the group consisting of mercaptocarboxylic acid, mercaptoaminoalkane, mercaptoaldehyde, dimercaptoalkane, and sulfide and disulfide having a reaction group such as carboxyl, thiol, alcohol, aldehyde, and amine; and the non-reactive linker having the non-reactive group is one selected from the group consisting of mercaptoalkane, mercaptoalcohol, sulfide, and disulfide.

25. The method of claim 24, wherein the linker having the reaction group is mercaptocarboxylic acid or mercaptoaminoalkane, and the non-reactive linker having the non-reactive group is mercaptoalcohol or mercaptoalkane.

26. The method of claim 24, wherein the linker having the reaction group is mercaptoaldehyde, and the non-reactive linker having the non-reactive group is mercaptoalcohol or mercaptoalkane.

27. The method of claim 24, wherein the linker having the reaction group is dimercaptoalkane, and the non-reactive linker having the non-reactive group is mercaptoalcohol or mercaptoalkane.

28. The method of claim 24, wherein the mercaptocarboxylic acid is 12-mercaptododecanoic acid.

29. The method of claim 24, wherein the mercaptoalcohol is 6-mercapto-1-hexanol and the mercaptoalkane is 1-heptanethiol.

30. The method of claim 2, wherein the linker having the reaction group is about 0.05 to about 50% of the total linker.

31. The method of claim 30, wherein the linker having the reaction group is about 0.05 to about 30% of the total linker.

32. The method of claim 1, which further comprises step (e) of removing the masking compound from the masked biologically active molecule immobilized in step (d).

33. A masked biologically active molecule immobilized on a supporting material made according to the method of any of claims 1 through 31.

34. A biologically active molecule immobilized on a supporting material made according to the method of claim 32.

35. The biologically active and immobilized molecule of claim 34, wherein the supporting material is a polymer, co-polymer, polymer blend, graft co-polymer or polymer adduct.

36. The biologically active and immobilized molecule of claim 35, wherein the supporting material is poly(ethylene glycol), poly(vinyl pyrrolidone), poly(vinyl alcohol), poly(amino acids), divinylether maleic anhydride, ethylene-maleic anhydride, N-(2-hydroxypropyl)methacrylamide, dextran; or a blend thereof.

37. A method for immobilizing a biologically active molecule having one or more active sites on a supporting (or substrate) material having a plurality of reactive linkers each having a reaction group comprising the steps of:
(c) combining the biologically active molecule with a masking compound that specifically binds to the active site to form a masked molecule; and
(d) immobilizing the masked molecule prepared in step (a) on the supporting (or substrate) material by reacting the molecule with the reaction groups, the reacting being under controlled conditions whereby the masked molecule binds to an average of less than about two of the reaction groups, wherein the number density of the reactive linker on the supporting material is adjusted to between about 2×10¹² cm⁻² to about 2×10¹⁴ cm⁻².

38. The method of claim 37, wherein the supporting (or substrate) material further comprises non-reactive linkers.

39. The method of claim 37, wherein the controlled conditions of step (b) further comprises forming a thin film of the reactive linker on the supporting material and controlling the mole fraction (or the number density) of the linker.

40. The method of claim 37, wherein the controlled conditions of step (b) further comprises controlling concentration of the masked biologically active molecule.

41. The method of claim 37, wherein the controlled conditions of step (b) further comprises adjusting at least one of reaction pH, time, and temperature.

42. The method of claim 37, which the controlled conditions of step (b) further comprises activating the reaction group of the linker by using a coupling reagent.

43. The method of claim 37, wherein the biologically active molecule is protein, enzyme, antigen, receptor, antibody; or biologically active fragment thereof.

44. The method of claim 37, wherein the masking compound is selected from the group consisting of substrate, antibody, antigen, ligand, inhibitor, cofactor, or a derivative, analogue or biologically active fragment thereof.

45. The method of claim 37, wherein the masking compound binds through covalent bonding, ionic bonding, coordination bonding, hydrogen bonding, dipole-dipole interaction, packing, or a combination thereof.

46. The method of claim 37, wherein the masking ratio of the biologically active molecule is between from about 5% to about 100%.

47. The method of claim 37, wherein the substrate material is metal, non-metal, semiconductor, an metallic or non-metallic oxide, organic or inorganic macromolecule, dendrimer, or a mixture thereof.

48. The method of claim 47, wherein the substrate material is planar, spherical, linear, porous, a microfabricated gel pad, or a nano-particle.

49. The method of claim 37, wherein the linker forms a thin film by covalent bonding, ionic bonding, coordination bonding, hydrogen bonding, packing, or combination thereof.

50. The method of claim 37, wherein the reaction group of the linker comprises a thiol, sulfide, disulfide, silane, carboxyl, amine, alcohol, aldehyde, epoxy, alkyl halide, alkene, alkyne, aryl, or a combination thereof.

51. The method of claim 37, wherein the reaction group of the linker that reacts with the biologically active molecule comprises carboxyl, amine, alcohol, aldehyde, epoxy, thiol, sulfide, disulfide, alkyl halide, alkene, alkyne, aryl, or a combination thereof.

52. The method of claim 37, wherein the biologically active molecule and the reaction group of the linker are connected by covalent bonding, ionic bonding, coordination bonding, hydrogen bonding, packing, or a combination thereof.

53. The method of claim 37, wherein the biologically active molecule and the reaction group of the linker are connected by amide bonding, imine bonding, sulfide bonding, disulfide bonding, ester bonding, ether bonding, amine bonding, or combination thereof.

54. The method of claim 51, wherein the amine group of the biologically active molecule and the carboxyl group of the linker are connected by amide bonding.

55. The method of claim 51, wherein the carboxyl group of the biologically active molecule and an amine reaction group of the linker are connected by amide bonding.

56. The method of claim 51, wherein an amine group of the biologically active molecule and an aldehyde reaction group of the linker are connected by imine bonding.

57. The method of claim 51, wherein an aldehyde group of the biologically active molecule and an amine reaction group of the linker are connected by imine bonding.

58. The method of claim 51, wherein a thiol group of the biologically active molecule and a thiol reaction group of the linker are connected by disulfide bonding.

59. The method of claim 39, wherein the linker having a reaction group is selected from the group consisting of mercaptocarboxylic acid, mercaptoaminoalkane, mercaptoaldehyde, dimercaptoalkane, sulfide, disulfide, carboxyl, thiol, alcohol, aldehyde, and amine.

60. The method of claim 39, wherein the controlling step further comprises adding to the substrate a non-reactive linker, the linker having a non-reactive group selected from the group consisting of mercaptoalkane, mercaptoalcohol, sulfide, and disulfide.

61. The method of claim 60, wherein the reactive linker having the reaction group is mercaptocarboxylic acid or mercaptoaminoalkane, and the non-reactive linker having the non-reactive group is mercaptoalcohol or mercaptoalkane.

62. The method of claim 60, wherein the linker having the reaction group is mercaptoaldehyde, and the non-reactive linker having the non-reactive group is mercaptoalcohol or mercaptoalkane.

63. The method of claim 60, wherein the linker having the reaction group is dimercaptoalkane, and the non-reactive linker having the non-reactive group is mercaptoalcohol or mercaptoalkane.

64. The method of claim 59, wherein the mercaptocarboxylic acid is 12-mercaptododecanoic acid.

65. The method of claim 60, wherein the mercaptoalcohol is 6-mercapto-1-hexanol and the mercaptoalkane is 1-heptanethiol.

66. The method of claim 60, wherein the linker having the reaction group is about 0.05% to about 50% of the total linker.

67. The method of claim 66, wherein the linker having the reaction group is about 0.05% to about 30% of the total linker.

68. The method of claim 37, wherein the method further comprises removing the masking compound from the masked biologically active molecule after immobilization.

69. A masked biologically active molecule immobilized on a supporting material made according to the method of claim 37.

70. A biologically active molecule immobilized on a supporting material made according to the method of claim 37.

71. The biologically active and immobilized molecule of claim 70, wherein the supporting material is a polymer, co-polymer, polymer blend, graft co-polymer or polymer adduct.

72. The biologically active and immobilized molecule of claim 71, wherein the supporting material is poly(ethylene glycol), poly(vinyl pyrrolidone), poly(vinyl alcohol), poly(amino acids), divinylether maleic anhydride, ethylene-maleic anhydride, N-(2-hydroxypropyl)methacrylamide, dextran; or a blend thereof.
